(19) United States
(12) Patent Application Publication
(10) Pub. No.: US 2025/0170319 A1
(43) Pub. Date: May 29, 2025

(54) VENOUS CANNULA INSERTION

(71) Applicant: RAMBAM MEDTECH LTD., Haifa (IL)

(72) Inventor: Roi GLAM, Zikhron Yaakov (IL)

(73) Assignee: RAMBAM MEDTECH LTD., Haifa (IL)

(21) Appl. No.: 18/834,339

(22) PCT Filed: Jan. 26, 2023
(86) PCT No.: PCT/IL2023/050090

§ 371 Date: Jul. 30, 2024

Related U.S. Application Data
(60) Provisional application No. 63/306,193, filed on Feb. 3, 2022.

(51) Int. Cl. A61M 1/36; A61M 1/16; A61M 25/10

(52) U.S. Cl. CPC A61M 1/3659; A61M 25/1011; A61M 1/1698; A61M 1/3666; A61M 2025/1097; A61M 2205/0216; A61M 2210/125

(57) ABSTRACT

Apparatus (20) is provided for use with an extracorporeal membrane oxygenation (ECMO) or cardiopulmonary bypass (CPB) machine. The apparatus (20) includes a venous cannula (22), which has a proximal end (24) and a distal tip (26), and is shaped so as to define (a) a blood-drainage lumen (28) through the venous cannula (22), and (b) at or near the distal tip (26), one or more openings (30) between the blood-drainage lumen (28) and outside the venous cannula (22). A connector (36) is coupled to the proximal end (24) of the venous cannula (22) in fluid communication with the blood-drainage lumen (28), and is configured to be coupled to the ECMO or CPB machine. A fluid-tight distal balloon (38) is coupled to the venous cannula (22) near the distal tip (26) of the venous cannula (22). An external proximal balloon (40) is configured to be disposed outside a subject's body. A balloon-connection lumen (42) couples the distal balloon (38) in fluid communication with the external proximal balloon (40). Other embodiments are also described.

### CROSS-REFERENCE TO RELATED APPLICATIONS

The present application claims priority from U.S. Provisional Application 63/306,193, filed Feb. 3, 2022, which is assigned to the assignee of the present application and incorporated herein by reference.

### FIELD OF THE APPLICATION

The present invention relates generally to extracorporeal life support, and specifically to techniques for venous cannulation for extracorporeal life support.

### BACKGROUND OF THE APPLICATION

Inserting a venous cannula while connecting a patient to an extracorporeal membrane oxygenation (ECMO) machine to support venoarterial (VA) or venovenous (VV) ECMO requires positioning the tip of the cannula at the right atrium to allow draining of venous blood from the heart. Conventionally, the location of the cannula is determined using transthoracic or transesophageal echocardiography, which is time-consuming and complex.

### SUMMARY OF THE APPLICATION

Embodiments of the present invention provide improved techniques for positioning a venous cannula, optionally without using radiography. The venous cannula comprises a distal balloon near a distal tip, and an external proximal balloon, which is configured to be disposed outside the subject's body. A balloon-connection lumen couples the distal balloon in fluid communication with the external proximal balloon.

To position the venous cannula, it is advanced through venous vasculature toward a right atrium while, during at least a portion of the advancing, pressure is applied to fluid contained within the external proximal balloon. Advancing of the venous cannula is ceased in response to a decrease in a volume of the fluid in the external proximal balloon that is indicative of transfer of some of the fluid from the external proximal balloon to the distal balloon and expansion of the distal balloon in the right atrium, which is wider than the venous vasculature. After the venous cannula is secured in place, the fluid is drained from the distal balloon, and blood flows through a blood-drainage lumen of the venous cannula.

These techniques generally allow quicker and simpler deployment of a venous cannula, typically without using any radiography during the procedure. At the same time, these techniques allow the surgeon full control over the procedure.

There is therefore provided, in accordance with an application of the present invention, apparatus for use with an extracorporeal membrane oxygenation (ECMO) or cardiopulmonary bypass (CPB) machine, the apparatus including:
a venous cannula, which has a proximal end and a distal tip, and is shaped so as to define:
   a blood-drainage lumen through the venous cannula, and
   at or near the distal tip, one or more openings between the blood-drainage lumen and outside the venous cannula;
a connector, which is coupled to the proximal end of the venous cannula in fluid communication with the blood-drainage lumen, and is configured to be coupled to the ECMO or CPB machine;
a distal balloon, which is fluid-tight and is coupled to the venous cannula near the distal tip of the venous cannula;
an external proximal balloon, which is configured to be disposed outside a body of a subject; and
a balloon-connection lumen, which couples the distal balloon in fluid communication with the external proximal balloon.

For some applications, the distal balloon is elastic.

For some applications, the elastic distal balloon is more compliant than the external proximal balloon.

For some applications, the external proximal balloon is elastic such that when the external proximal balloon contains fluid, the external proximal balloon applies pressure to the fluid contained therewithin.

For some applications, an outer diameter of the venous cannula is 8-29 Fr.

For some applications, a distance (a) between a distal end of the distal balloon and (b) the distal tip is no more than 2 cm.

For some applications, the distance is 1-2 cm.

For some applications, a distance between (a) a proximal end of the distal balloon and (b) the distal tip is no more than 7 cm.

For some applications, the distance is 3-5 cm.

For some applications, the balloon-connection lumen runs at least partially along the venous cannula.

For some applications, the connector includes a vented or non-vented 0.95 cm connector.

For some applications, the venous cannula is wire wound.

For some applications, the apparatus further includes one or more springs arranged to apply external pressure to an external surface of the external proximal balloon.

For some applications, when the distal balloon is unconstrained and inflated at 10 cm H2O, a greatest external dimension of the distal balloon, measured perpendicular to a central longitudinal axis of the venous cannula, is 5-7 cm.

For some applications, when the distal balloon is unconstrained and inflated at 10 cm H2O, a greatest external dimension of the distal balloon, measured perpendicular to a central longitudinal axis of the venous cannula, equals 200%-500% of an outer diameter of the venous cannula.

For some applications, when the distal balloon is unconstrained and inflated at 10 cm H2O, a volume of the distal balloon is 3-30 ml.

For some applications, when the external proximal balloon is unconstrained and inflated at 10 cm H2O, a volume of the external proximal balloon is 50-250 ml.

For some applications, the venous cannula has an outer diameter of 8-29 Fr.

For some applications, the venous cannula has an insertable length of 10-50 cm.

For some applications, the distal balloon at least partially surrounds the venous cannula.

For some applications, the distal balloon entirely surrounds the venous cannula near the distal tip of the venous cannula.

For some applications, the distal balloon partially surrounds the venous cannula near the distal tip of the venous cannula.

For some applications, when the external proximal balloon is unconstrained and inflated with an external-proximal-balloon volume of fluid at 10 cm H2O, and the distal balloon is unconstrained and inflated with a distal-balloon volume of fluid at 10 cm H2O:
the external-proximal-balloon volume is no less than the distal-balloon volume.

For some applications, the external-proximal-balloon volume equals at least 125% of the distal-balloon volume.

For some applications, the external-proximal-balloon volume equals at least 200% of the distal-balloon volume.

For some applications:
distal and proximal ends of the distal balloon are sealed to an external surface of a wall of the venous cannula at respective distal and proximal balloon sites along the venous cannula,
the one or more openings include one or more balloon-coinciding lateral openings through the wall of the venous cannula at respective lateral-opening sites that are longitudinally between the distal and proximal balloon sites, and
the apparatus is configured such that the distal balloon, when not inflated, does not occlude the one or more balloon-coinciding lateral openings.

For some applications:
a wall of the distal balloon is shaped so as to define one or more balloon-wall lateral openings through the wall of the distal balloon, and
the wall of the distal balloon is sealed to the external surface of the wall of the venous cannula around respective borders of at least a portion of the one or more balloon-coinciding lateral openings, such that the one or more balloon-wall lateral openings are in fluid communication with the blood-drainage lumen via the at least a portion of the one or more balloon-coinciding lateral openings, respectively.

For some applications, the distal balloon entirely surrounds the venous cannula near the distal tip of the venous cannula.

For some applications, the distal balloon partially surrounds the venous cannula near the distal tip of the venous cannula, such that at least a portion of the one or more balloon-coinciding lateral openings are not covered by the distal balloon.

For some applications, the apparatus is configured such that upon inflation of the distal balloon, when unconstrained, to at least a threshold pressure, the distal balloon occludes at least one of the one or more balloon-coinciding lateral openings.

There is further provided, in accordance with an application of the present invention, a method of inserting a venous cannula into a body of a subject, the method including:
inserting a distal tip of the venous cannula into venous vasculature of the subject, wherein a fluid-tight distal balloon is coupled to the venous cannula near the distal tip;
advancing the venous cannula through the venous vasculature toward a right atrium while, during at least a portion of the advancing, pressure is applied to fluid contained within an external proximal balloon that is disposed outside the subject's body and is coupled in fluid communication with the distal balloon via a balloon-connection lumen;
while the pressure is applied to the fluid contained within the external proximal balloon, ceasing advancing the venous cannula in response to a decrease in a volume of the fluid in the external proximal balloon that is indicative of transfer of some of the fluid from the external proximal balloon to the distal balloon and expansion of the distal balloon in the right atrium;
after ceasing the advancing, draining the distal balloon;
connecting the venous cannula to an extracorporeal membrane oxygenation (ECMO) or cardiopulmonary bypass (CPB) machine; and
thereafter, activating the ECMO or CPB machine.

For some applications, the distal balloon is elastic.

For some applications, the elastic distal balloon is more compliant than the external proximal balloon.

For some applications, the external proximal balloon is elastic such that the external proximal balloon applies the pressure to the fluid contained therewithin.

For some applications, advancing the venous cannula through the venous vasculature while, during the at least a portion of the advancing, the pressure is applied to the fluid contained within the external proximal balloon includes:
advancing the venous cannula through the venous vasculature while, during the at least a portion of the advancing, external pressure is applied to an external surface of the external proximal balloon so as to apply the pressure to the fluid contained within the external proximal balloon.

For some applications, advancing the venous cannula through the venous vasculature while, during the at least a portion of the advancing, the external pressure is applied to the external surface of the external proximal balloon includes:
advancing the venous cannula through the venous vasculature while, during the at least a portion of the advancing, manually applying the external pressure to the external surface of the external proximal balloon.

For some applications, advancing the venous cannula through the venous vasculature while, during the at least a portion of the advancing, the external pressure is applied to the external surface of the external proximal balloon includes:
advancing the venous cannula through the venous vasculature while, during the at least a portion of the advancing, the external pressure is applied to the external surface of the external proximal balloon by one or more springs.

For some applications, advancing the venous cannula through the venous vasculature while, during the at least a portion of the advancing, the external pressure is applied to the external surface of the external proximal balloon, includes:
advancing the venous cannula through a proximal portion of the venous vasculature while the external pressure is not applied to the external surface of the external proximal balloon; and
thereafter, advancing the venous cannula through a distal portion of the venous vasculature while the external pressure is applied to the external surface of the external proximal balloon.

For some applications, advancing the venous cannula through the venous vasculature while, during the at least a portion of the advancing, the external pressure is applied to the external surface of the external proximal balloon, includes:
advancing the venous cannula through a proximal portion of the venous vasculature while a first amount of the external pressure is applied to the external surface of the external proximal balloon; and
thereafter, advancing the venous cannula through a distal portion of the venous vasculature while a second amount of the external pressure is applied to the external surface of the external proximal balloon, the second amount greater than the first amount.

For some applications, ceasing advancing the venous cannula in response to the decrease in the volume of the fluid in the external proximal balloon includes ceasing advancing the venous cannula upon manually sensing the decrease in the volume of the fluid in the external proximal balloon.

For some applications, inserting the distal tip of the venous cannula into the venous vasculature includes inserting the distal tip of the venous cannula into the venous vasculature while the distal balloon contains some of the fluid.

For some applications, inserting the distal tip includes inserting the distal tip into the venous vasculature while the distal balloon is deflated.

For some applications, the method further includes, after ceasing the advancing of the venous cannula in response to the decrease in a volume of the fluid in the external proximal balloon, proximally withdrawing the venous cannula until the distal tip is located at a junction between a central vein of the venous vasculature and the right atrium.

For some applications, the method further includes, after ceasing the advancing of the venous cannula in response to the decrease in a volume of the fluid in the external proximal balloon, fixating the venous cannula to skin of the subject.

The present invention will be more fully understood from the following detailed description of embodiments thereof, taken together with the drawings, in which:

### BRIEF DESCRIPTION OF THE DRAWINGS

FIGS. 1A-B are schematic illustrations of apparatus for use with an extracorporeal membrane oxygenation (ECMO) or cardiopulmonary bypass (CPB) machine, in accordance with an application of the present invention:

FIGS. 2A-B are schematic illustrations of a distal portion of a venous cannula of the apparatus of FIGS. 1A-B, in accordance with an application of the present invention;

FIGS. 3A-D are schematic illustration of a method of inserting the venous cannula of the apparatus of FIGS. 1A-B into a subject's body, in accordance with an application of the present invention; and

FIG. 4 is a schematic illustration of an alternative configuration of the apparatus of FIGS. 1A-B, in accordance with an application of the present invention.

### DETAILED DESCRIPTION OF APPLICATIONS

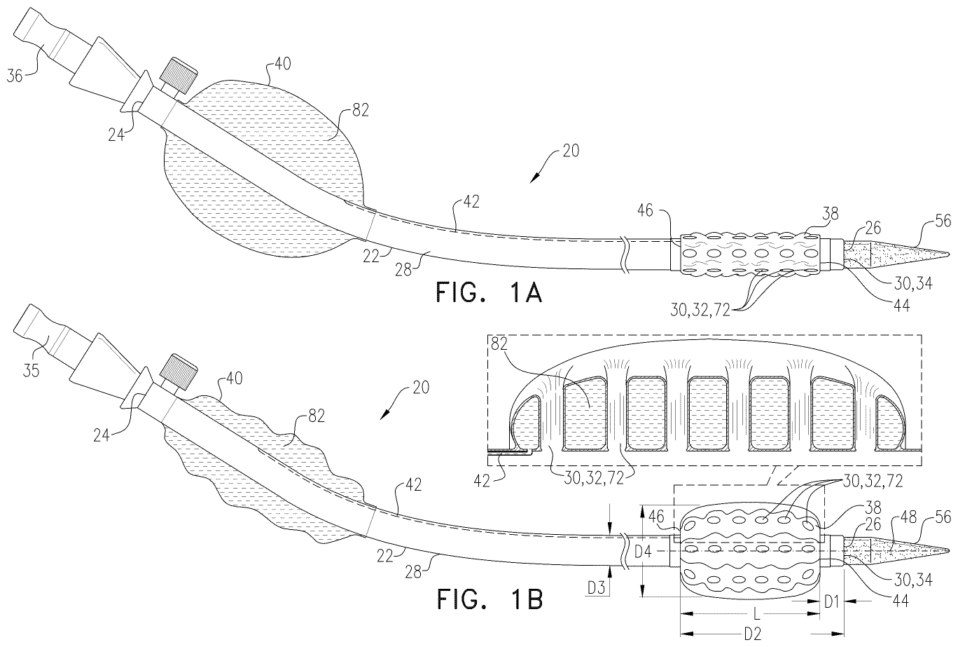

FIGS. 1A-B are schematic illustrations of apparatus 20 for use with an extracorporeal membrane oxygenation (ECMO) or cardiopulmonary bypass (CPB) machine, in accordance with an application of the present invention. (An ECMO machine is also known as an ECMO circuit or as extracorporeal life support (ECLS), and a CPB machine is also known as a heart-lung machine.)

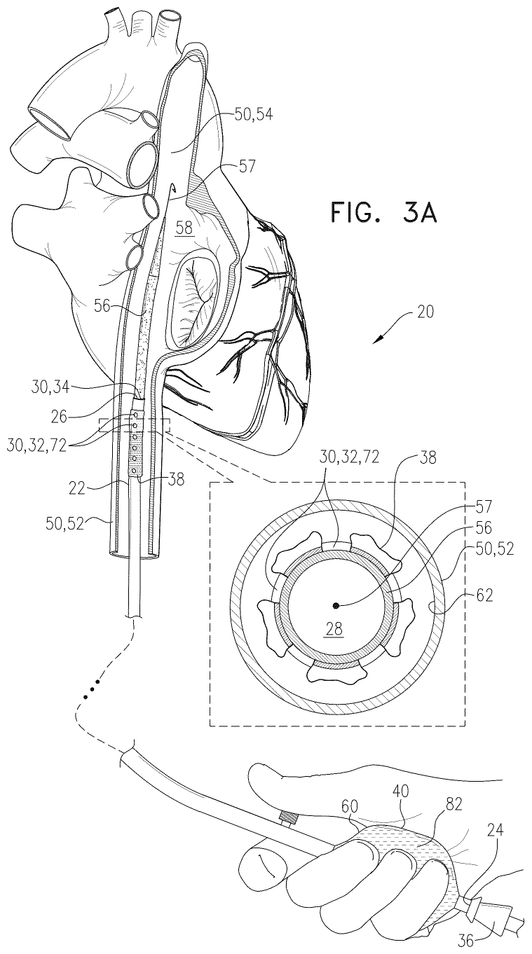

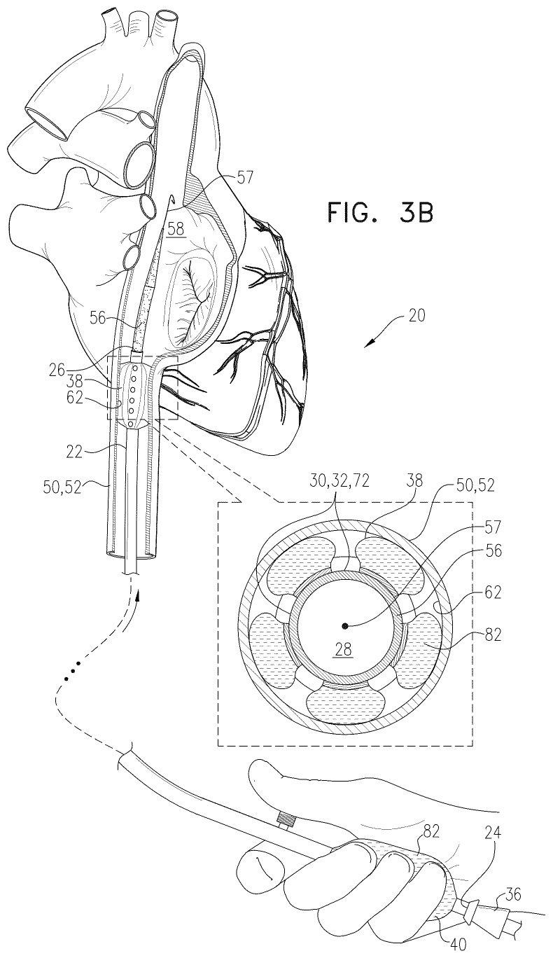

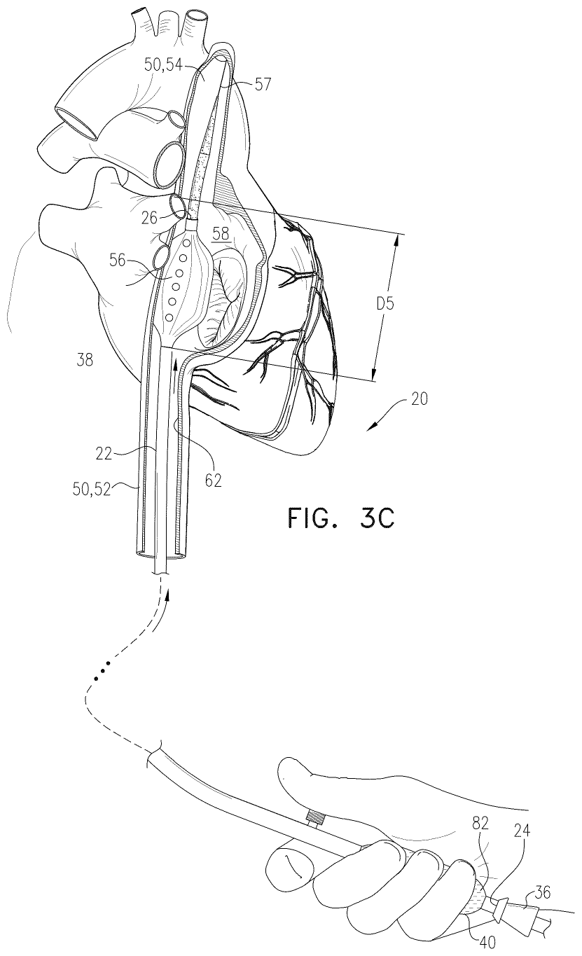

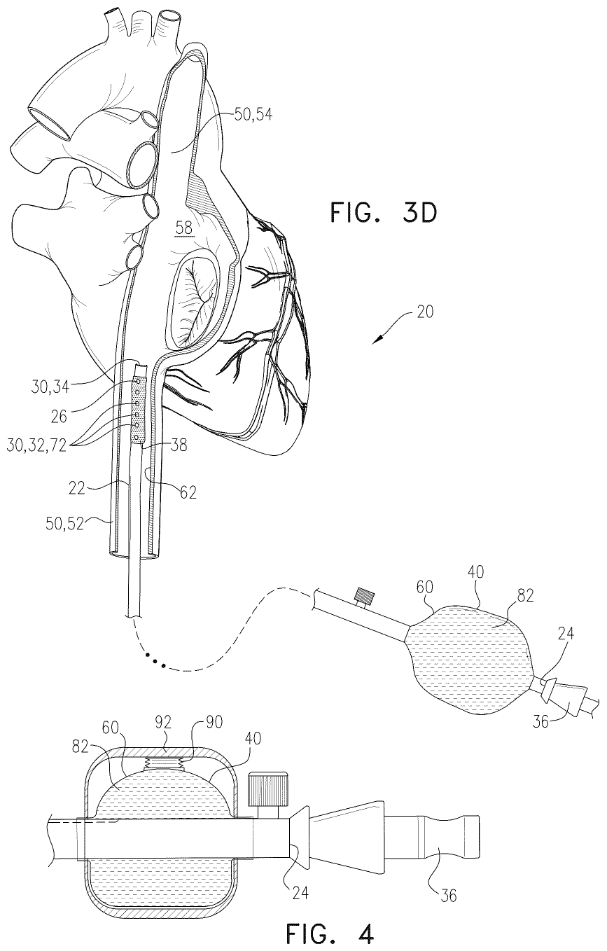

Apparatus 20 comprises a venous cannula 22, which has a proximal end 24 and a distal tip 26. Venous cannula 22 is shaped so as to define a blood-drainage lumen 28 through venous cannula 22. Venous cannula 22 is also shaped so as to define, at or near distal tip 26, one or more openings 30 between blood-drainage lumen 28 and outside venous cannula 22. Typically, the one or more openings 30 include one or more lateral openings 32 and a distal opening 34. (Distal opening 34, in addition to providing drainage, also permits passage of an introducer 56 and/or a guidewire 57, such as described hereinbelow with reference to FIG. 3A.)

Apparatus 20 further comprises:
a connector 36, which is coupled to proximal end 24 of venous cannula 22 in fluid communication with blood-drainage lumen 28, and is configured to be coupled to the ECMO or CPB machine; optionally, connector 36 comprises a vented or non-vented 0.95 cm connector (generally referred to in the art as a ⅜″ connector);
a distal balloon 38, which is fluid-tight and is coupled to venous cannula 22 near distal tip 26 of venous cannula 22; typically, but not necessarily, distal balloon 38 is elastic;
an external proximal balloon 40, which is configured to be disposed outside a body of a subject; and
a balloon-connection lumen 42, which couples distal balloon 38 in fluid communication with external proximal balloon 40, and typically runs at least partially along venous cannula 22 (either inside (as shown), outside (configuration not shown), embedded in a wall of the cannula, or a combination of two or more of these locations).

For some applications, external proximal balloon 40 is elastic. In some applications in which both external proximal balloon 40 and distal balloon 38 are elastic, elastic distal balloon 38 is more compliant than external proximal balloon 40.

FIG. 1A shows apparatus 20 in an exemplary resting state, after filling of external proximal balloon 40 with fluid 82, and without application of external pressure to an external surface 60 of external proximal balloon 40, such as described hereinbelow with reference to FIG. 3A. FIG. 1B shows apparatus 20 in an exemplary state after application of the external pressure to external surface 60 of external proximal balloon 40, such as described hereinbelow with reference to FIG. 3B.

For some applications, distal balloon 38 at least partially surrounds venous cannula 22 near distal tip 26 of venous cannula 22. For some of these applications, distal balloon 38 entirely surrounds venous cannula 22, such as shown in the drawings. For others of these applications, distal balloon 38 partially surrounds venous cannula 22 (configuration not shown). As used in the present application, including in the claims, “surrounds” the venous cannula means extends around a circumference of the venous cannula, but not necessarily entirely around the circumference.

For some applications, apparatus 20 comprises a plurality of distal balloons 38 coupled to venous cannula 22 near distal tip 26, typically at least partially surrounding venous cannula 22 (configuration not shown). Distal balloon 38 are in fluid communication with external proximal balloon 40, via balloon-connection lumen 42 or a plurality of balloon-connection lumens 42.

For some applications, external proximal balloon 40 is directly coupled to venous cannula 22, typically near a proximal end of venous cannula 22. For some of these applications, external proximal balloon 40 at least partially surrounds venous cannula 22 near the proximal end of venous cannula 22. For some of these applications, external proximal balloon 40 entirely surrounds venous cannula 22, such as shown in the drawings. For others of these applications, external proximal balloon 40 partially surrounds venous cannula 22 (configuration not shown).

External proximal balloon 40 is typically no smaller than distal balloon 38, such as larger than distal balloon 38. More particularly, for some applications, when external proximal balloon 40 is unconstrained (by anatomy of the subject or otherwise) and inflated with an external-proximal-balloon volume of fluid 82 at 10 cm H2O, and distal balloon 38 is unconstrained and inflated with a distal-balloon volume of fluid 82 at 10 cm H2O: the external-proximal-balloon volume is no less than the distal-balloon volume, such as at least 125%, e.g., at least 200%, of the distal-balloon volume.

(This comparison may be performed both when external proximal balloon 40 and distal balloon 38 are in fluid communication with each other or when they are not in fluid communication with each other.)

As used in the present application, including in the claims, the volume of distal balloon 38 is the amount of free space enclosed by distal balloon 38, excluding any space within distal balloon 38 occupied by venous cannula 22. Similarly, as used in the present application, including in the claims, the volume of external proximal balloon 40 is the amount of free space enclosed by external proximal balloon 40, excluding any space within external proximal balloon 40 occupied by venous cannula 22.

Venous cannula 22 is flexible and typically kink-resistant. Typically, to provide the kink-resistance, venous cannula 22 is wire wound to provide radial structural support, as is known in the cannula art.

Reference is still made to FIGS. 1A-B. For some applications, a distance D1 (a) between a distal end 44 of distal balloon 38 and (b) distal tip 26 is no more than 2 cm, such as 0.5-2 cm, e.g., 1-2 cm, such as for positioning distal tip 26 in an inferior vena cava (IVC) 52. For other applications, the distance D1 is at least 0.5 cm, e.g., 0.5-2 cm, such as for positioning distal tip 26 in a superior vena cava (SVC) 54.

For some applications, a distance D2 between (a) a proximal end 46 of distal balloon 38 and (b) distal tip 26 is no more than 7 cm, such as 2-7 cm, e.g., 4-5 cm, both for positioning distal tip 26 in IVC 52 or in SVC 54.

For some applications, a length L between 44 and 46 is 60%-90% of a distance D5 between the orifice of IVC 52 and the orifice of SVC 54 (labeled in FIG. 3C), such as 70%-90%, e.g., 75%-85%, such as 80%, of the distance D4.

For some applications, venous cannula 22 has an outer diameter D3 of 8-29 Fr, such as, for example, 8, 10, 12, 14, 15, 17, 19, 21, 23, 25, 27, or 29 Fr.

For some applications, venous cannula 22 has an insertable length of 10-50 cm.

For some applications, when distal balloon 38 is unconstrained (by anatomy of the subject or otherwise) and inflated at 10 cm H2O:
a volume of distal balloon 38 is at least 3 ml, no more than 30 ml, and/or 3-30 ml (the size of distal balloon 38 may be selected based on the patient's age and/or gender),
a greatest external dimension D4 of distal balloon 38, measured perpendicular to a central longitudinal axis 48 of venous cannula 22, is at least 3 cm (e.g., at least 5cm, such as at least 6 cm), no more than 7 cm (e.g., no more than 6 cm), and/or 3-7 cm, e.g., 5-7 cm, 3-5 cm, or 5-6 cm, and/or
the greatest external dimension D4 of distal balloon 38, measured perpendicular to central longitudinal axis 48 of venous cannula 22, equals at least 200%, no more than 500%, and/or 200%-500% of the outer diameter D3 of venous cannula 22.

In configurations in which distal balloon 38 has a circular cross-section at its widest point, the greatest external dimension D4 is the external diameter of the balloon.

For some applications, when distal balloon 38 is uninflated, a wall 76 thereof extends radially outward from an external surface 64 of a wall 66 of venous cannula 22 by no more than 2 mm, typically no more than 1 mm, e.g., 0.5-1 mm.

For some applications, when external proximal balloon 40 is unconstrained (by anatomy of the subject or otherwise) and inflated at 10 cm H2O, a volume of external proximal balloon 40 is at least 50 ml, no more than 250 ml, and/or 50-250 ml.

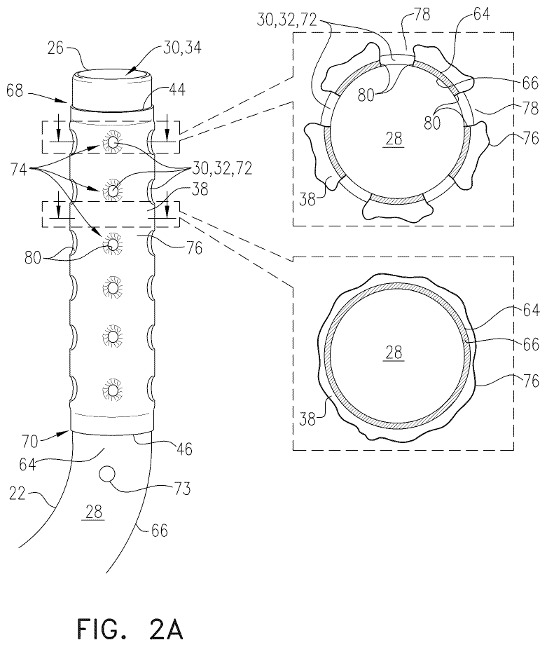

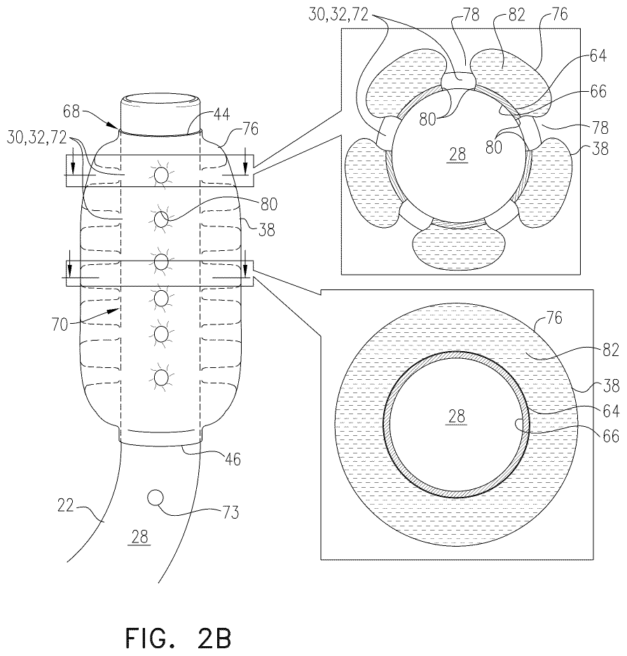

Reference is still made to FIGS. 1A-B, and is additionally made to FIGS. 2A-B, which are schematic illustrations of a distal portion of venous cannula 22, in accordance with an application of the present invention. FIGS. 2A and 2B show distal balloon 38 uninflated and inflated, respectively.

In this configuration, distal and proximal ends 44 and 46 of distal balloon 38 are sealed to external surface 64 of wall 66 of venous cannula 22 at respective distal and proximal balloon sites 68 and 70 along venous cannula 22. The one or more lateral openings 32 include one or more balloon-coinciding lateral openings 72 through wall 66 of venous cannula 22 at respective lateral-opening sites 74 (labeled in FIG. 2A) that are longitudinally between distal and proximal balloon sites 68 and 70. Apparatus 20 is configured such that distal balloon 38, when not inflated, such as shown in FIG. 2A, does not occlude the one or more balloon-coinciding lateral openings 72.

Distal balloon 38, when inflated, such as shown in FIG. 2B, may or may not occlude the one or more balloon-coinciding lateral openings 72; in FIG. 2B, distal balloon 38 is shown, by way of example and not limitation, as not occluding the one or more balloon-coinciding lateral openings 72. Occlusion would only be temporary during the placement procedure. For some applications, apparatus 20 is configured such that upon inflation of distal balloon 38, when unconstrained, to at least a threshold pressure (and/or at least a threshold volume), distal balloon 38 occludes at least one of the one or more balloon-coinciding lateral openings 72.

For some applications, the one or more lateral openings 32 further include one or more non-balloon-coinciding lateral openings 73 through wall 66 of venous cannula 22 at respective lateral-opening sites that are not longitudinally between distal and proximal balloon sites 68 and 70. For other applications, the one or more lateral openings 32 do not include any non-balloon-coinciding lateral openings 73.

For some applications, wall 76 of distal balloon 38 is shaped so as to define one or more balloon-wall lateral openings 78 through wall 76 of distal balloon 38. Wall 76 of distal balloon 38 is sealed to external surface 64 of wall 66 of venous cannula 22 around respective borders 80 of at least a portion of the one or more balloon-coinciding lateral openings 72, such that the one or more balloon-wall lateral openings 78 are in fluid communication with blood-drainage lumen 28 via the at least a portion of the one or more balloon-coinciding lateral openings 72, respectively. For some of these applications, distal balloon 38 entirely surrounds venous cannula 22 near the distal tip of venous cannula 22, such as shown, while for others of these applications, distal balloon 38 partially surrounds venous cannula 22 near the distal tip of venous cannula 22, such that at least a portion of the one or more balloon-coinciding lateral openings 72 are not covered by distal balloon 38 (configuration not shown).

For some applications:
venous cannula 22 comprises polyurethane,
distal balloon 38 comprises PVC, rubber, or silicone, and/or
external proximal balloon 40 comprises PVC, rubber, or silicone.

Reference is now made to FIGS. 3A-D, which are schematic illustration of a method of inserting venous cannula 22 into the subject's body, in accordance with an application of the present invention.

The method comprises inserting distal tip 26 of venous cannula 22 into venous vasculature of the subject, using insertion (e.g., cannulation) techniques known in the art, such as percutaneous cannulation of the femoral vein or the jugular vein using the Seldinger technique. Typically, distal balloon 38 is deflated, or only slightly inflated (i.e., contains some of fluid 82), during the insertion. Typically, venous cannula 22 is introduced with the aid of introducer 56 that is disposed within blood-drainage lumen 28 of venous cannula 22 and extending several centimeters out of distal opening 34 at distal tip 26, as is known the cannula art. Introducer 56 is typically advanced over guidewire 57, as known in the art.

Optionally, as a safety check, before insertion of venous cannula 22 into the venous vasculature, distal balloon 38 is filled by applying pressure to external proximal balloon 40, in order to ensure that the two balloons are connects and the system is safe for use.

As shown in FIG. 3A, venous cannula 22 is advanced through the venous vasculature into a central vein 50 and toward a right atrium 58 while, during at least a portion of the advancing, external pressure is applied to external surface 60 of external proximal balloon 40, which is disposed outside the subject's body and contains fluid 82, typically a liquid such as a physiological solution, e.g., saline solution. For example, central vein 50 may be IVC 52 in deployments in which venous cannula 22 is introduced by percutaneous cannulation of the femoral vein, such as shown, or SVC 54 in deployments in which venous cannula 22 is introduced by percutaneous cannulation of the jugular vein (approach not shown). External proximal balloon 40 typically has been partially or entirely filled with fluid 82 before the advancing, typically before insertion of venous cannula 22 into the venous vasculature, using techniques known in the art, such as injection from a syringe through a septum or one-way valve.

As used in the present application, including in the claims, application of “external pressure” means application of pressure to external surface 60 of external proximal balloon 40 from outside external proximal balloon 40, rather than application of internal pressure, such as, for example, via balloon-connection lumen 42 by the squeezing of distal balloon 38.

The external pressure may optionally be applied to external surface 60 of external proximal balloon 40 only during a portion of the advancing of venous cannula 22 through the venous vasculature toward right atrium 58, such as only after venous cannula 22 is near right atrium 58, as ascertained by the physician based on the length of venous cannula 22 inserted into the venous vasculature and the known approximate distance along the venous vasculature to the right atrium. Alternatively, a lower level of external pressure may be applied during advancement through a proximal portion of the venous vasculature than during advancement through a subsequent distal portion of the venous vasculature (including central vein 50). Avoiding premature, unnecessary application of the external pressure may help reduce the possible risk of damage to the wall of the venous vasculature by distal balloon 38 if expanded throughout the advancing.

For some applications, such as shown in FIGS. 3A-C, the external pressure is manually applied to external surface 60 of external proximal balloon 40, such as by the physician performing the procedure or another healthcare worker. For some other applications, the external pressure is applied to external surface 60 of external proximal balloon 40 by an element of apparatus 20, such as one or more springs, such as described hereinbelow with reference to FIG. 4.

As described above, external proximal balloon 40 is coupled in fluid communication with distal balloon 38 via balloon-connection lumen 42 (shown in FIGS. 1A-B). As shown in FIG. 3B, the external pressure applied to external surface 60 of external proximal balloon 40 causes some of fluid 82 to be transferred to distal balloon 38, thereby inflating distal balloon 38. However, the level of inflation, and thus the amount of fluid 82 transferred, is limited by the internal diameter of a wall 62 of central vein 50 of the venous vasculature.

As shown in FIG. 3C, while the external pressure is applied to external surface 60 of external proximal balloon 40, when distal balloon 38 reaches right atrium 58, which is substantially wider than central vein 50, distal balloon 38 is less constrained, such as not constrained, from expansion. As a result, the external pressure applied to external surface 60 of external proximal balloon 40 transfers additional fluid 82 from external proximal balloon 40 to distal balloon 38.

This transfer of fluid 82 from external proximal balloon 40 to distal balloon 38 reduces the volume of fluid 82 in external proximal balloon 40 and results in the contraction of external proximal balloon 40 (and the expansion of distal balloon 38). The physician performing the procedure ceases advancing venous cannula 22 in response to the decrease in the volume of fluid 82 in external proximal balloon 40 that is indicative of transfer of some of fluid 82 from external proximal balloon 40 to distal balloon 38 and expansion of distal balloon 38 in right atrium 58. For example, the decrease in the volume of fluid 82 may be ascertained by sensing manually, sensing with a gauge, visually observing, and/or a combination of techniques. Optionally, apparatus 20 comprises a sensor and a user output that is configured to alert the physician upon detecting a threshold reduction in the volume of fluid 82 in external proximal balloon 40, such as by an audio, visual, and/or audiovisual signal.

After the advancing is ceased:
venous cannula 22 is fixated, typically to skin of the subject; and
before or after venous cannula 22 is fixated, distal balloon 38 is drained, so as to deflate the distal balloon, such as shown in FIG. 3D.

For example, the elasticity of distal balloon 38 may expel fluid 82 from distal balloon 38 once the external pressure is no longer applied to external surface 60 of external proximal balloon 40. Alternatively, for example, a syringe is used to manually drain distal balloon 38, or a negative pressure is formed in external proximal balloon 40 to drain distal balloon 38.

Typically, venous cannula 22 is connected to an ECMO machine (also known as an ECMO circuit) or a CPB machine (also known as a heart-lung machine), via connector 36.

Reference is made to FIG. 3D. Optionally, after ceasing advancement of venous cannula 22 in response to the decrease in the volume of fluid 82 in external proximal balloon 40, and before fixation of venous cannula 22, venous cannula 22 is proximally withdrawn until distal tip 26 is located at a junction 84 between central vein 50 (e.g., IVC 52) and right atrium 58. For example, the venous cannula may be proximally withdrawn by a predetermined distance, e.g., 3 cm. Distal balloon 38 may be drained before or after being proximally withdrawn.

Alternatively, the method does not include this additional step, and distal tip 26 is instead left in place in right atrium 58. The location of distal tip 26 within right atrium, including how close the tip is to SVC 54, depends on the distance D1 between distal end 44 of distal balloon 38 and distal tip 26.

Further alternatively, the distance D1 between distal end 44 of distal balloon 38 and distal tip 26 is sufficiently large (e.g., 0.5-2 cm) that distal tip 26 is disposed within SVC 54 (configuration not shown).

Typically, after placement of venous cannula 22 as described above, and connection of venous cannula 22 to the ECMO or CPB machine, the ECMO or CPB machine is activated.

Reference is now made to FIG. 4, which is a schematic illustration of an alternative configuration of apparatus 20, in accordance with an application of the present invention. In this configuration, the external pressure is applied to external surface 60 of external proximal balloon 40 by an element of apparatus 20, such as one or more springs 90.

Alternatively, in configurations in which external proximal balloon 40 is elastic, external proximal balloon 40 may apply pressure to fluid 82 contained therein. In these configurations:
at the step of the method described hereinabove with reference to FIG. 3A, pressure is applied to fluid 82 by elastic external proximal balloon 40, typically without external pressure being applied to external surface 60 of external proximal balloon 40,
at the step of the method described hereinabove with reference to FIG. 3B, the pressure applied to fluid 82 by elastic external proximal balloon 40 causes some of fluid 82 to be transferred to distal balloon 38, thereby inflating distal balloon 38, and
at the step of the method described hereinabove with reference to FIG. 3C, the pressure applied to fluid 82 by elastic external proximal balloon 40 transfers additional fluid 82 from external proximal balloon 40 to distal balloon 38.

Alternatively, pressure is applied to fluid 82 contained within external proximal balloon 40 by another element other than external proximal balloon 40, in which case external proximal balloon 40 is not necessarily elastic.

In some applications of the present invention, venous cannula 22 comprises a dual-lumen cannula, as is known in the art.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for use with an extracorporeal membrane oxygenation (ECMO) or cardiopulmonary bypass (CPB) machine, the apparatus comprising:
a venous cannula, which has a proximal end and a distal tip, and is shaped so as to define:
a blood-drainage lumen through the venous cannula, and
at or near the distal tip, one or more openings between the blood-drainage lumen and outside the venous cannula;
a connector, which is coupled to the proximal end of the venous cannula in fluid communication with the blood-drainage lumen, and is configured to be coupled to the ECMO or CPB machine;
a distal balloon, which is fluid-tight and is coupled to the venous cannula near the distal tip of the venous cannula;
an external proximal balloon, which is configured to be disposed outside a body of a subject; and
a balloon-connection lumen, which couples the distal balloon in fluid communication with the external proximal balloon.

2. The apparatus according to claim 1, wherein the distal balloon is elastic.

3. The apparatus according to claim 2, wherein the elastic distal balloon is more compliant than the external proximal balloon.

4. The apparatus according to claim 1, wherein the external proximal balloon is elastic such that when the external proximal balloon contains fluid, the external proximal balloon applies pressure to the fluid contained therewithin.

5. The apparatus according to claim 1, wherein an outer diameter of the venous cannula is 8-29 Fr.

6. The apparatus according to claim 1, wherein a distance (a) between a distal end of the distal balloon and (b) the distal tip is no more than 2 cm.

7. The apparatus according to claim 6, wherein the distance is 1-2 cm.

8. The apparatus according to claim 1, wherein a distance between (a) a proximal end of the distal balloon and (b) the distal tip is no more than 7 cm.

9. The apparatus according to claim 8, wherein the distance is 3-5 cm.

10. The apparatus according to claim 1, wherein the balloon-connection lumen runs at least partially along the venous cannula.

11. The apparatus according to claim 1, wherein the connector comprises a vented or non-vented 0.95 cm connector.

12. The apparatus according to claim 1, wherein the venous cannula is wire wound.

13. The apparatus according to claim 1, further comprising one or more springs arranged to apply external pressure to an external surface of the external proximal balloon.

14. The apparatus according to claim 1, wherein, when the distal balloon is unconstrained and inflated at 10 cm H2O, a greatest external dimension of the distal balloon, measured perpendicular to a central longitudinal axis of the venous cannula, is 5-7 cm.

15. The apparatus according to claim 1, wherein when the distal balloon is unconstrained and inflated at 10 cm H2O, a greatest external dimension of the distal balloon, measured perpendicular to a central longitudinal axis of the venous cannula, equals 200%-500% of an outer diameter of the venous cannula.

16. The apparatus according to claim 1, wherein when the distal balloon is unconstrained and inflated at 10 cm H2O, a volume of the distal balloon is 3-30 ml.

17. The apparatus according to claim 1, wherein, when the external proximal balloon is unconstrained and inflated at 10 cm H2O, a volume of the external proximal balloon is 50-250 ml.

18. The apparatus according to claim 1, wherein the venous cannula has an outer diameter of 8-29 Fr.

19. The apparatus according to claim 1, wherein the venous cannula has an insertable length of 10-50 cm.

20. The apparatus according to any one of claims 1-19, wherein the distal balloon at least partially surrounds the venous cannula.

21. The apparatus according to claim 20, wherein the distal balloon entirely surrounds the venous cannula near the distal tip of the venous cannula.

22. The apparatus according to claim 20, wherein the distal balloon partially surrounds the venous cannula near the distal tip of the venous cannula.

23. The apparatus according to any one of claims 1-19, wherein when the external proximal balloon is unconstrained and inflated with an external-proximal-balloon volume of fluid at 10 cm H2O, and the distal balloon is unconstrained and inflated with a distal-balloon volume of fluid at 10 cm H2O:
the external-proximal-balloon volume is no less than the distal-balloon volume.

24. The apparatus according to claim 23, wherein the external-proximal-balloon volume equals at least 125% of the distal-balloon volume.

25. The apparatus according to claim 24, wherein the external-proximal-balloon volume equals at least 200% of the distal-balloon volume.

26. The apparatus according to any one of claims 1-19,
wherein distal and proximal ends of the distal balloon are sealed to an external surface of a wall of the venous cannula at respective distal and proximal balloon sites along the venous cannula,
wherein the one or more openings include one or more balloon-coinciding lateral openings through the wall of the venous cannula at respective lateral-opening sites that are longitudinally between the distal and proximal balloon sites, and
wherein the apparatus is configured such that the distal balloon, when not inflated, does not occlude the one or more balloon-coinciding lateral openings.

27. The apparatus according to claim 26,
wherein a wall of the distal balloon is shaped so as to define one or more balloon-wall lateral openings through the wall of the distal balloon, and
wherein the wall of the distal balloon is sealed to the external surface of the wall of the venous cannula around respective borders of at least a portion of the one or more balloon-coinciding lateral openings, such that the one or more balloon-wall lateral openings are in fluid communication with the blood-drainage lumen via the at least a portion of the one or more balloon-coinciding lateral openings, respectively.

28. The apparatus according to claim 27, wherein the distal balloon entirely surrounds the venous cannula near the distal tip of the venous cannula.

29. The apparatus according to claim 26, wherein the distal balloon partially surrounds the venous cannula near the distal tip of the venous cannula, such that at least a portion of the one or more balloon-coinciding lateral openings are not covered by the distal balloon.

30. The apparatus according to claim 26, wherein the apparatus is configured such that upon inflation of the distal balloon, when unconstrained, to at least a threshold pressure, the distal balloon occludes at least one of the one or more balloon-coinciding lateral openings.

31. A method of inserting a venous cannula into a body of a subject, the method comprising:
inserting a distal tip of the venous cannula into venous vasculature of the subject, wherein a fluid-tight distal balloon is coupled to the venous cannula near the distal tip;
advancing the venous cannula through the venous vasculature toward a right atrium while, during at least a portion of the advancing, pressure is applied to fluid contained within an external proximal balloon that is disposed outside the subject's body and is coupled in fluid communication with the distal balloon via a balloon-connection lumen;
while the pressure is applied to the fluid contained within the external proximal balloon, ceasing advancing the venous cannula in response to a decrease in a volume of the fluid in the external proximal balloon that is indicative of transfer of some of the fluid from the external proximal balloon to the distal balloon and expansion of the distal balloon in the right atrium;
after ceasing the advancing, draining the distal balloon;
connecting the venous cannula to an extracorporeal membrane oxygenation (ECMO) or cardiopulmonary bypass (CPB) machine; and
thereafter, activating the ECMO or CPB machine.
